(19) United States
(12) Patent Application Publication
(10) Pub. No.: US 2022/0270745 A1
(43) Pub. Date: Aug. 25, 2022

(54) ULTRASOUND DIAGNOSTIC APPARATUS AND ULTRASOUND EXAMINATION ASSISTING METHOD

(71) Applicant: FUJIFILM Healthcare Corporation, Kashiwa-shi (JP)

(72) Inventor: Yuko SATO, Chiba (JP)

(21) Appl. No.: 17/672,820

(22) Filed: Feb. 16, 2022

(30) Foreign Application Priority Data
Feb. 22, 2021 (JP) 2021-026000

(51) Int. Cl. G16H 30/20

(52) U.S. Cl. CPC G16H 30/20

(57) ABSTRACT

A protocol corrector corrects standard protocol information, to generate specialized protocol information for a particular subject of examination. A specialized protocol execution controller controls execution of a sequence of processes of a specialized protocol based on the specialized protocol information. A reference image and a measurement value are associated with each process of the specialized protocol.

### CROSS REFERENCE TO RELATED APPLICATION

This application claims priority to Japanese Patent Application No. 2021-026000 filed on Feb. 22, 2021, which is incorporated herein by reference in its entirety including the specification, claims, drawings, and abstract.

### TECHNICAL FIELD

The present disclosure relates to an ultrasound diagnostic apparatus and a method of assisting ultrasound examination, and in particular to generation or management of an examination protocol including a plurality of processes (steps or stages) which are sequentially executed.

### BACKGROUND

In an ultrasound examination, normally, a sequence of processes are sequentially executed according to a procedure which is determined in an examination guideline or the like. For example, in an ultrasound examination of an abdominal region, in each process, a predetermined cross section of a diagnosis target organ is displayed as a tomographic image. On each tomographic image, necessary measurement is executed. At the end of each process, a process execution result (such as a tomographic image, a measurement result, a finding, or the like) is stored.

In order to assist an examiner during execution of the sequence of processes, various examination protocols are prepared as examination assisting functions in the ultrasound diagnostic apparatus. For example, when a particular examination protocol is selected and executed, an execution condition which is set in advance is automatically set for each process. In addition, during execution of the process, a necessary measurement is automatically started up. When a store operation is executed, the procedure automatically transitions to the next process. Each individual process of the examination protocol is also called a “view”, from the viewpoint of image observation.

Document 1 (JP 2010-259614 A) discloses an ultrasound diagnostic apparatus in which an optimum examination protocol for a subject of examination is applied to the subject of examination. The optimum examination protocol is an examination protocol which was applied to the subject of examination in the past. Document 2 (JP 2020-141883 A) discloses an ultrasound diagnostic apparatus in which, when a new examination processing is executed during execution of a certain examination protocol, the new examination processing is registered as a branch protocol. Document 3 (JP 2018-61659 A) discloses an ultrasound diagnostic apparatus in which an examination protocol is generated based on an image array. Documents 1 to 3 do not disclose an ultrasound diagnostic apparatus in which an examination protocol for a particular subject of examination is generated from a general-purpose examination protocol, and these two examination protocols can be selectively used.

From the viewpoint of assisting the ultrasound examination for various subjects of examination, it is necessary to prepare a general-purpose or standard examination protocol, but, with the preparation of the standard examination protocol alone, it is not possible to sufficiently assist the ultrasound examination. In some cases, a parameter may be changed according to the subject of examination during a certain process during execution of the examination protocol. In other cases, a new process may be added when an abnormal site is found during the execution of the examination protocol. If the examination protocol which is customized in these manners can be re-used in later times, it becomes possible to more efficiently execute a next ultrasound examination for the same subject of examination.

An advantage of the present disclosure lies in provision of an ultrasound diagnostic apparatus which can assist an ultrasound examination according to a subject of examination. Alternatively, an advantage of the present disclosure lies in realizing a system which enables exploitation of a past setting content during execution of an examination protocol. Alternatively, an advantage of the present disclosure lies in enabling coexistence of general-purpose usability and specialized usability in the use of the examination protocol.

### SUMMARY

According to one aspect of the present disclosure, there is provided an ultrasound diagnostic apparatus comprising: a protocol corrector that corrects standard protocol information for executing a standard protocol which is a standard examination protocol, to generate specialized protocol information for executing a specialized protocol which is an examination protocol for a particular subject of examination; and a protocol execution controller that, when the standard protocol is selected, sequentially sets a plurality of execution conditions based on the standard protocol information during a course of sequential execution of a sequence of processes of the standard protocol, and that, when the specialized protocol is selected, sequentially sets a plurality of execution conditions based on the specialized protocol information during a course of sequential execution of a sequence of processes of the specialized protocol.

According to another aspect of the present disclosure, there is provided a method of assisting ultrasound examination, the method comprising: correcting standard protocol information for executing a standard protocol which is a standard examination protocol, to generate specialized protocol information for executing a specialized protocol which is an examination protocol for a particular subject of examination; sequentially setting a plurality of execution conditions based on the standard protocol information during a course of sequential execution of a sequence of processes of the standard protocol, when the standard protocol is selected; and sequentially setting a plurality of execution conditions based on the specialized protocol information and displaying reference information acquired from the particular subject of examination and associated with the specialized protocol, during a course of sequential execution of a sequence of processes of the specialized protocol, when the specialized protocol is selected.

### BRIEF DESCRIPTION OF DRAWINGS

Embodiment(s) of the present disclosure will be described based on the following figures, wherein:

FIG. 1 is a block diagram showing an ultrasound diagnostic apparatus according to an embodiment of the present disclosure;

FIG. 2 is a diagram showing an example of standard protocol information;

FIG. 3 is a diagram showing an example of specialized protocol information;

FIG. 4 is diagram showing a plurality of standard protocol management tables;

FIG. 5 is a diagram showing a plurality of specialized protocol management tables;

FIG. 6 is a diagram showing an example display;

FIG. 7 is a diagram showing a measurement value graph;

FIG. 8 is a flowchart showing an example operation; and

FIG. 9 is a diagram showing a specific example of S24.

### DESCRIPTION OF EMBODIMENTS

An embodiment of the present disclosure will now be described with reference to the drawings.

### (1) Overview of Embodiment

An ultrasound diagnostic apparatus according to an embodiment of the present disclosure comprises a protocol corrector and a protocol execution controller. The protocol corrector corrects standard protocol information for executing a standard protocol which is a standard examination protocol, to generate specialized protocol information for executing a specialized protocol which is an examination protocol for a particular subject of examination. The protocol execution controller sequentially sets a plurality of execution conditions based on the standard protocol information during a course of sequential execution of a sequence of processes of the standard protocol, when the standard protocol is selected, and sequentially sets a plurality of execution conditions based on the specialized protocol information during a course of sequential execution of a sequence of processes of the specialized protocol, when the specialized protocol is selected. A processor to be described below functions as the protocol corrector and the protocol execution controller.

According to the above-described configuration, the protocol corrector corrects the standard protocol information, and specialized protocol information is generated. When executing a protocol, the standard protocol and the specialized protocol may be selectively used. For a new subject of examination, the standard protocol may be applied. For a subject of examination for which the specialized protocol is generated, the specialized protocol may be applied so that the ultrasound examination of the subject of examination can be more appropriately assisted.

In the correction of the standard protocol, a parameter which is input or designated by the examiner during a course of examination of a plurality of processes of the standard protocol may be used. For example, when a diagnosis depth range determined as a default value in the standard protocol is changed by the examiner, there is generated a specialized protocol which reflects the diagnosis depth after the change.

According to an embodiment of the present disclosure, the protocol corrector has a reference image association unit which associates, with respect to the sequence of processes of the specialized protocol and as a plurality of reference images, a plurality of ultrasound images acquired by the execution of the sequence of processes. The protocol execution controller has a reference image display controller which executes, during the course of sequential execution of the sequence of processes of the specialized protocol, display control to sequentially display the plurality of reference images associated with the sequence of processes. According to this structure, a plurality of ultrasound images acquired from a particular subject of examination in the past can be stepwise displayed during the ultrasound examination of the present time. By referring to the ultrasound images, a position and an orientation of a probe can be easily re-produced. In addition, it becomes easier to understand a change of properties and characteristics of an affected site.

According to an embodiment of the present disclosure, the protocol corrector has a measurement value association unit which associates, with respect to a process with measurement in the specialized protocol, a measurement value acquired by execution of the process with measurement. The protocol execution controller has a measurement value display controller which executes, during execution of the process with measurement in the specialized protocol, display control to display one or a plurality of measurement values of the past associated with the process with measurement. According to this structure, the measurement of the present time can be executed while referring to the measurement value(s) acquired in the past. When the measurement value is acquired, the acquired measured value can be easily compared with the measurement value acquired in the past.

According to an embodiment of the present disclosure, the measurement value display controller has a function to execute the display control to display the plurality of measurement values of the past as a measurement value graph. According to this structure, a change with respect to time of the measurement value can be easily understood.

An ultrasound diagnostic apparatus according to an embodiment of the present disclosure comprises an examination value association unit that associates, with respect to the specialized protocol, a plurality of examination values acquired in a time sequential order from the particular subject of examination. The protocol execution controller has an examination value display controller which executes, during execution of the specialized protocol, display control to display an examination value graph showing the plurality of examination values associated with the specialized protocol. According to this structure, ultrasound examination can be performed in consideration of a change with respect to time of the examination value.

According to an embodiment of the present disclosure, the standard protocol information includes a plurality of sets of process information for executing the plurality of processes of the standard protocol. The protocol corrector has a function to correct content of a set of process information which is a correction target among the plurality of sets of process information, and a function to add new sets of process information to the plurality of sets of process information. Alternatively, the protocol corrector may have a function to delete a process, a function to copy a process, or the like.

A method of assisting ultrasound examination according to an embodiment of the present disclosure comprises a first step, a second step, and a third step. In the first step, standard protocol information for executing a standard protocol which is a standard examination protocol is corrected, to generate specialized protocol information for executing a specialized protocol which is an examination protocol for a particular subject of examination. In the second step, when the standard protocol is selected, during a course of sequential execution of a sequence of processes of the standard protocol, a plurality of execution conditions are sequentially set based on the standard protocol information. In the third step, when the specialized protocol is selected, during a course of sequential execution of a sequence of processes of the specialized protocol, a plurality of execution conditions are sequentially set based on the specialized protocol information, and reference information which is information acquired from the particular subject of examination and which is associated with the specialized protocol is displayed.

According to the above-described structure, the standard protocol or the specialized protocol can be selectively used according to the subject of examination. When the specialized protocol is selected, an examiner assisting process based on a past setting record may be performed. Alternatively, a particular specialized protocol may be automatically selected based on the subject of examination. Alternatively, a particular specialized protocol may be automatically selected based on a combination of the subject of examination and an examination site (or diagnosis item). Alternatively, other information such as probe position information may be linked to each process. In the following, in some cases, the standard protocol and the specialized protocol will be collectively referred to as a protocol.

### (2) Details of Embodiment

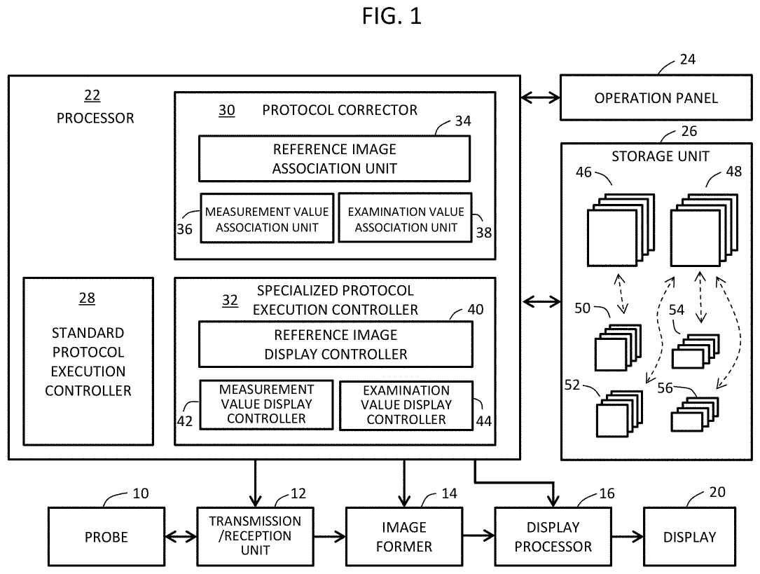

FIG. 1 shows an ultrasound diagnostic apparatus according to an embodiment of the present disclosure. The ultrasound diagnostic apparatus is a medical apparatus which is placed in a medical institution and used during an ultrasound examination.

A probe 10 transmits and receives ultrasound. In the probe 10, a transducer array formed from a plurality of transducers is provided. An ultrasound beam is formed by the transducer array, and is electronically scanned. A beam scanning plane is formed by this process.

A transmission/reception unit 12 supplies a plurality of transmission signals in parallel to each other to the transducer array during the transmission, and applies phase-alignment and summing (that is, delay and summing) on a plurality of receptions signals from the transducer array during reception. With these processes, beam data are formed. With one scanning of the ultrasound beam, one set of reception frame data is generated. Each set of reception frame data is formed from a plurality of sets of beam data arranged in an electron scanning direction. Each individual set of beam data is formed from a plurality of sets of echo data arranged in a depth direction.

The reception frame data which are sequentially output from the transmission/reception unit 12 are sequentially input to an image former 14. The image former 14 includes a DSC (Digital Scan Converter). The DSC generates a display frame data array from a reception frame data array. The DSC has a coordinate conversion function, a pixel interpolation function, or the like. One display frame data forms one set of tomographic image data. The ultrasound diagnostic apparatus shown in FIG. 1 further includes other image formers which form other ultrasound images (for example, a two-dimensional bloodstream image).

A display processor 16 has an image combining function, a color processing function, or the like. The display processor 16 forms a display image to be displayed on a display 20. The display image includes an ultrasound image. The ultrasound image is a video image in a real time operation, and is a still image at a time of freeze. The display 20 is formed from an LCD, an organic EL display, or the like. The display image includes various images generated by a processor 22, which may include a process list image, a measurement list image, a measurement value graph, an examination value graph, or the like.

The processor 22 is formed from a CPU which executes a program. The processor 22 controls operations of various elements in the ultrasound diagnostic apparatus, and also executes necessary information processing. In FIG. 1, a plurality of functions of the processor 22 are represented by a plurality of blocks.

A standard protocol execution controller 28 controls execution of a standard protocol according to standard protocol information defining the standard protocol. The standard protocol is a protocol which is generally used, and is formed from a plurality of processes which are sequentially executed.

A protocol corrector 30 corrects the standard protocol information, to generate specialized protocol information defining a specialized protocol. The protocol corrector 30 also corrects the specialized protocol information, to generate other specialized protocol information. The specialized protocol is a protocol for a particular subject of examination, and is formed from a plurality of processes. The standard protocol and the specialized protocol are both examination protocols, and do not differ from each other in substance. The protocols differ from each other in that, while the standard protocol is a general-purpose, typical protocol for unspecified subjects of examination, the specialized protocol is a customized protocol for a particular subject of examination.

A specialized protocol execution controller 32 controls execution of the specialized protocol according to the specialized protocol information defining the specialized protocol. The standard protocol execution controller 28 and the specialized protocol execution controller 32 are basically identical in substance. In FIG. 1, in order to distinguish the two protocols, the standard protocol execution controller 28 and the specialized protocol execution controller 32 are separately represented.

During a course of execution of the protocol, at a start of execution of each process, an execution condition determined for the process is set in the ultrasound diagnostic apparatus. The execution condition includes parameters such as an operation mode, a diagnosis depth, a transmission frequency, a transmission repetition period, a gain, or the like. The operation mode includes, for example, a B mode, a CFM mode, a PW mode, or the like. In the case of a process with measurement, in general, measurement is executed on a still image displayed after a freeze operation. As the measurement, there may be exemplified a distance measurement, an area measurement, a volume measurement, an index calculation, or the like,

Normally, when a store operation (that is, a save operation) is performed, the procedure proceeds to the next process. When the store operation is performed in a state in which a video image is being displayed, the video image is stored, and, when a store operation is performed in a state in which a still image is being displayed, the still image is stored. The stored image becomes a part of an examination report. Normally, with the storage of the image, the measurement value and an annotation are also stored.

During a course of execution of the protocol, more specifically, in the execution of each individual process, the examiner may change an arbitrary parameter as necessary. For example, a diagnosis depth range may be changed. In this case, upon completion of the execution of the protocol, a new specialized protocol is generated. With this process, the new specialized protocol can be used in a next ultrasound examination. When a basic parameter such as the operation mode is changed in the execution of a certain process, the execution of the protocol is temporarily interrupted, and a practically new process is additionally executed. After completion of this process, the execution of the protocol is restarted. In this case also, a new specialized protocol including the added process is generated by the protocol corrector 30 at the completion of the execution of the protocol.

In the illustrated example structure, the protocol corrector 30 comprises a reference image association unit 34, a measurement value association unit 36, and an examination value association unit 38. The reference image association unit 34 associates, as a reference image, the image stored in each individual process to the process. The measurement value association unit 36 associates a measurement value acquired in each individual process to the process. The examination value association unit 38 associates an examination value acquired from the subject of examination to the specialized protocol. As the examination value, there may be exemplified a height, a weight, a BMI, a blood analysis result, or the like. Alternatively, positional information of the probe may be associated with respect to each process.

In the present disclosure, a value acquired by measurement with respect to an ultrasound image will be referred to as a “measurement value”, and a value acquired by examination other than the ultrasound examination will be referred to as an “examination value”. Thus, the measurement value is an ultrasound measurement value, and the examination value is a non-ultrasound measurement value.

In the illustrated example structure, the specialized protocol execution controller 32 comprises a reference image display controller 40, a measurement value display controller 42, and an examination value display controller 44. The reference image display controller 40 executes control to display, in the execution of each individual process, a reference image associated with the process (an ultrasound image acquired during a previous examination) on a screen. When the process is switched, the content of the reference image is also switched.

The measurement value display controller 42 displays, in the execution of each individual process, the measurement value associated with the process (a measurement value acquired during a previous examination) as a numerical value. When a plurality of measurement values are associated with respect to the structure in a time sequential order, a measurement value graph may be generated based on these values, and may be displayed. That is, the measurement value display controller 42 has a graph generation function.

The examination value display controller 44 displays, during the execution of the protocol, the examination value associated with the protocol as a numerical value. When a plurality of examination values are associated with the protocol in a time sequential order, an examination value graph may be generated based on these values, and may be displayed. That is, the examination value display controller 44 has a graph generation function.

A storage unit 26 is formed from a semiconductor memory, a hard disk drive, or the like. The storage unit 26 stores a group of standard protocol information 46 and a group of specialized protocol information 48. The group of standard protocol information 36 is formed from a plurality of sets of standard protocol information corresponding to a plurality of diagnosis sites or to a plurality of diagnosis items. The group of specialized protocol information 48 is formed from a plurality of sets of specialized protocol information corresponding to a plurality of subjects of examination.

The storage unit 26 also stores a group of standard images 50, a group of reference images 52, a group of measurement values 54, and a group of examination values 56. The group of standard images 50 includes a plurality of standard images displayed during the execution of a plurality of processes of each standard protocol. Each individual standard image is formed from an ultrasound image of another person, a schema drawing, or the like, and is not an image acquired from the subject of the examination himself/herself.

The group of reference images is formed from a plurality of ultrasound images acquired from each subject of examination. In the course of protocol execution in the present time, a sequence of ultrasound images acquired in the previous protocol execution are sequentially displayed.

The group of measurement values 54 is formed from a plurality of measurement values acquired from each subject of examination. In the course of the protocol execution in the present time, a sequence of measurement values acquired in the previous protocol execution are sequentially displayed. As described above, a graph display in place of the numerical value display is also possible. The group of examination values 56 is formed from a plurality of examination values acquired from each subject of examination. In the course of the protocol execution in the present time, examination values acquired currently or in the past may be displayed. As described above, the graph display in place of the numerical value display is also possible.

An operation panel 24 is connected to the processor 22. The operation panel 24 is formed from a plurality of switches, a plurality of knobs, a keyboard, a trackball, or the like.

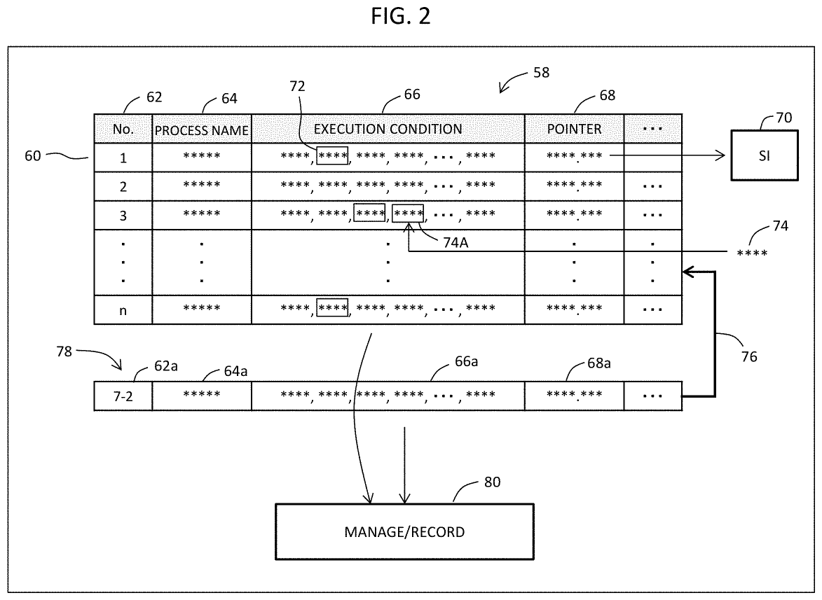

FIG. 2 shows an example of the standard protocol information. Standard protocol information 58 is information necessary when the execution of the standard protocol is to be controlled. The standard protocol includes a plurality of processes, and the standard protocol information 58 includes a plurality of sets of process information 60 corresponding to the plurality of processes. Each individual set of process information 60 includes a process number 62, a process name 64, an execution condition 66, a standard image pointer 68, or the like. The execution condition 66 is formed from a plurality of parameters defining a process operation. The standard image pointer 68 is link information for specifying a standard image 70 associated with the process, and is, for example, a URL. At the start of execution of each process, the standard image associated with the process is displayed. A position and an orientation of the probe may be adjusted while comparing the standard image and the ultrasound image.

In the course of execution of an arbitrary process, any of the parameters may be changed. For example, the diagnosis depth range may be changed. In FIG. 2, a plurality of parameters corrected by the examiner are surrounded by a plurality of boxes 72. For example, a particular parameter is replaced with a parameter 74 after change (refer to reference numeral 74A), and the standard protocol is corrected. The content of the registered standard protocol itself is maintained.

When the operation mode is changed or another basic parameter is changed in the course of the execution of an arbitrary process, the execution of the standard protocol is temporarily interrupted, and a practical additional process is executed. Reference numeral 78 shows process information 78 corresponding to the additional process. The process information 78 includes a process number 62a, a process name 64a, an execution condition 66a, a standard image pointer 68a, or the like. When the ultrasound image is stored in the additional process, the stored image is managed as a reference image. The standard image pointer 68a is information for specifying a location of the standard image. Reference numeral 76 shows a position at which the additional process is inserted.

The protocol corrector manages and records a parameter correction and a process addition (refer to reference numeral 80). Based on the recorded information, a specialized protocol for the subject of examination for which the ultrasound examination is currently performed is generated.

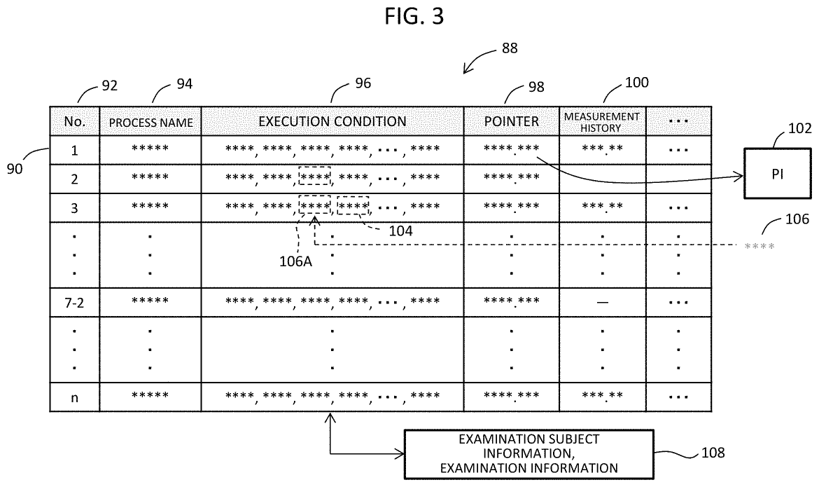

FIG. 3 shows an example of the specialized protocol information. Specialized protocol information 88 is information necessary when the execution of the specialized protocol is to be controlled. The specialized protocol is formed from a plurality of processes, and the specialized protocol information 88 includes a plurality of sets of process information 90 corresponding to the plurality of processes. Each individual set of process information 90 includes a process number 92, a process name 94, an execution condition 96, a reference image pointer 98, a measurement history 100, or the like. The execution condition 96 is formed from a plurality of parameters defining the process operation. The reference image pointer 98 is link information for specifying a reference image 102 associated with the process (image acquired during a previous examination), and is, for example, a URL. At a start of the execution of each process, a reference image associated with the process is displayed. The position and the orientation of the probe may be adjusted while comparing the reference image and the ultrasound image. In addition, reading of the ultrasound image (including follow-up observation) can be performed while referring to the reference image.

It is also possible to correct the specialized protocol information. A plurality of parameters surrounded by a plurality of boxes 104 are corrected parameters. For example, a parameter 104A is replaced with an original parameter 106. Alternatively, a process may be added.

To the specialized protocol information 88, examination subject information and examination information are associated (refer to reference numeral 108). The examination subject information includes an ID, a name, a gender, an age, an examination site, or the like. The examination information is formed from a group of examination values acquired by examinations other than the ultrasound examination.

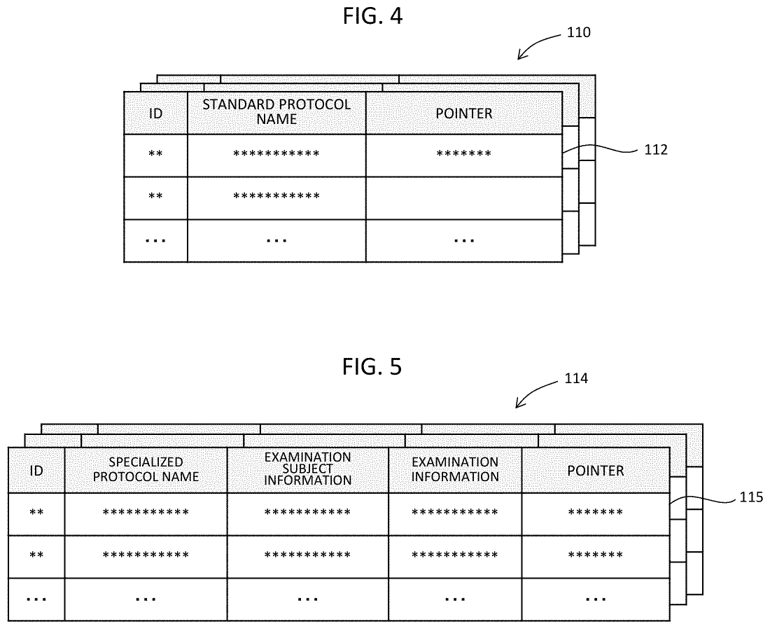

FIG. 4 shows an example management of a group of standard protocol information 110. For example, a plurality of tables 112 corresponding to a plurality of diagnosis items are formed, and a plurality of standard protocols are managed by each individual table 112. In the illustrated example structure, for each individual standard protocol, an ID, a standard protocol name, and a pointer are managed. The location of the standard protocol information is specified by the pointer.

FIG. 5 shows an example management of a group of specialized protocol information 114. Here, a plurality of tables 115 corresponding to a plurality of subjects of examination are formed. A plurality of specialized protocols are managed by each individual table 115. In the illustrated example structure, for each individual specialized protocol, an ID, a specialized protocol name, the examination subject information, the examination information, and a pointer are managed. The location of the specialized protocol information is specified by the pointer. By forming such a table 115, it becomes possible to quickly identify, for each subject of examination, one or a plurality of sets of specialized protocol information which can be applied to the subject of examination.

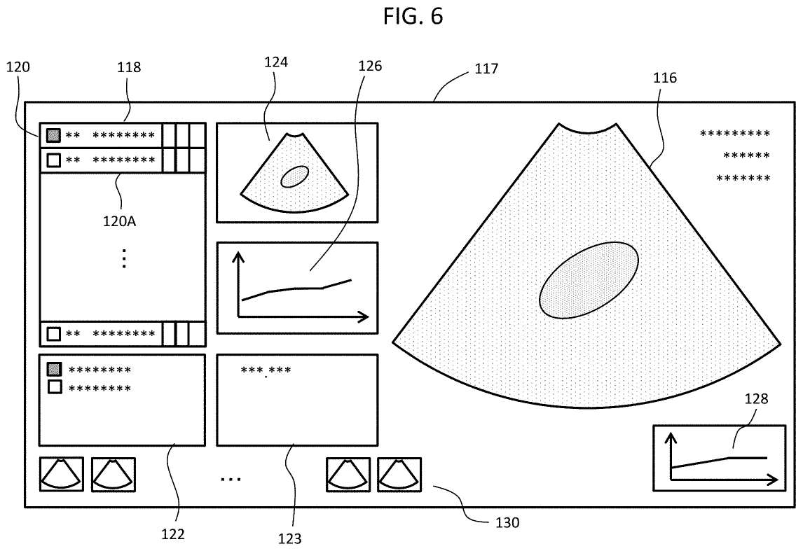

FIG. 6 shows an example display. A layout shown in FIG. 6 is merely an example layout, and various layouts corresponding to the situations and needs may be employed.

A display image 117 includes an ultrasound image 116. The ultrasound image 116 is, for example, a tomographic image. When a particular protocol is selected, a process list 118 showing the content of the particular protocol is displayed. The process list 118 is formed from a plurality of process displays 120 corresponding to the plurality of processes. A process display corresponding to a process 120A currently being executed is displayed in an identifiable manner. On a measurement list 122, information (such as measurement name) for specifying one or a plurality of measurements defined for the process currently being executed is displayed. In a measurement value display field 123, an acquired measurement value is displayed.

During a course of execution of the specialized protocol, from a start of the execution of each process, a reference image 124 associated with the process is displayed. The reference image is an ultrasound image of the same type, acquired from the same subject of examination. The position and the orientation of the probe may be adjusted while referring to the reference image. Further, the content of the ultrasound image 116 may be evaluated while referring to the reference image.

In addition, during the course of execution of the specialized protocol, during execution of each process, a measurement value graph 126 may be displayed as necessary. The measurement value graph 126 shows a plurality of measurement values in time sequential order, acquired from the same subject of examination. When there is only one measurement value, the measurement value may be displayed as a numerical value, or as a graph along with a measurement value acquired in the present time.

Further, in the course of execution of the specialized protocol, during execution of each process, an examination value graph 128 may be displayed as necessary. The examination value graph 128 shows a plurality of examination values in time sequential order, acquired from the same subject of examination. When there is only one examination value, the measurement value is displayed as a numerical value. Alternatively, a plurality of examination value graphs may be displayed. At a lower part of the display image 117, a plurality of thumbnail images 130 showing a plurality of stored ultrasound images are included.

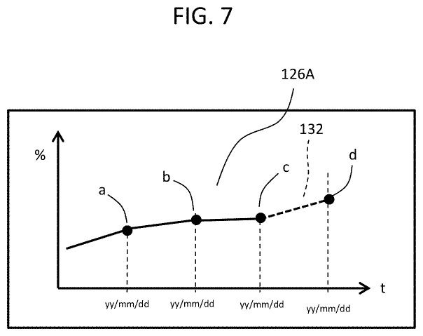

FIG. 7 exemplifies the measurement value graph. A measurement value graph 126A is formed from a plurality of measurement values a˜d. A horizontal axis is a time axis, and a vertical axis shows a size of the measurement value. The measurement values a˜c are values acquired in the past, and the measurement value d is acquired at the current time. In this manner, by reflecting the currently acquired measurement value d in the measurement value graph 126A, it becomes easier to perform the follow-up observation. Reference numeral 132 shows a graph portion which is extended by the plot of the measurement value d.

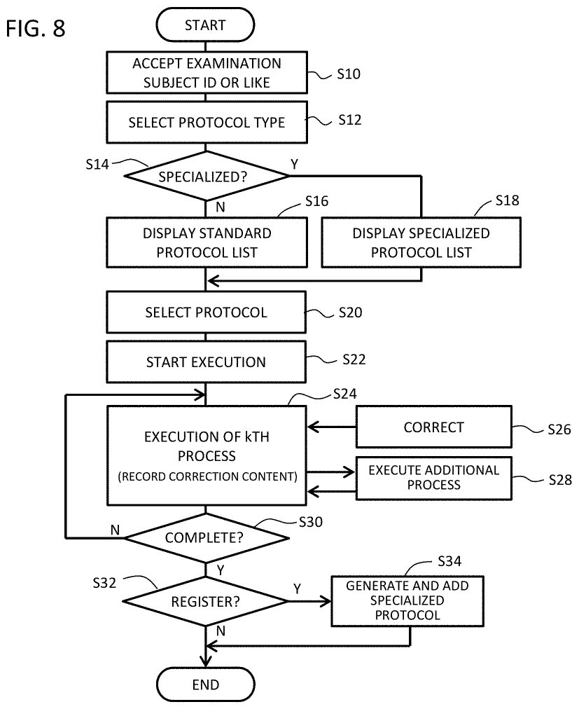

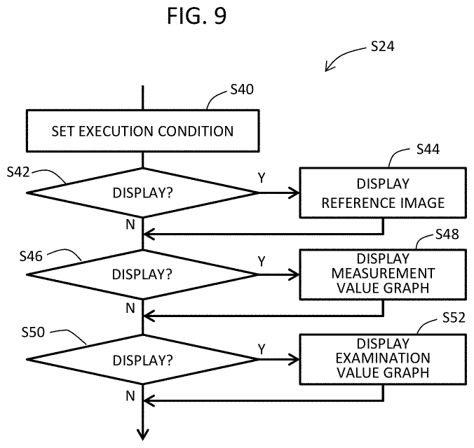

With reference to FIGS. 8 and 9, an example operation of the ultrasound diagnostic apparatus will now be described, and a method of assisting ultrasound examination according to an embodiment of the present disclosure will be summarized.

In S10, an examination subject ID or the like is input and is accepted. In S12, a protocol type is selected by the examiner. More specifically, a standard protocol or a specialized protocol is selected. In S14, the selected protocol type is determined. When the standard protocol is selected, in S16, the standard protocol list is displayed. When the specialized protocol is selected, in S18, the specialized protocol list is displayed. In S20, a particular protocol is selected by the examiner.

When it is possible to uniquely specify the specialized protocol correlated to the examination subject ID, the specialized protocol may be automatically selected after the step of S10. Alternatively, the specialized protocol may be automatically selected based on a combination of the examination subject ID and other information (for example, examination site information).

In S22, execution of the selected particular protocol is started. Specifically, the processes are sequentially executed in the order from a top process. This is shown in S24. Here, assuming k=1, 2, 3, . . . , a kth process is executed. When there is a correction of the parameter (S26), the correction is recorded, and, when an additional process is executed (S28), the content of the additional process is recorded.

In S30, it is judged whether or not execution of a final process is completed. When there remains a process which is yet to be executed, S24 is repeated. In S32, when the examiner selects YES in response to an inquiry of whether or not to register the new specialized protocol reflecting the correction content, in S34, a new specialized protocol is generated, and is registered. When the examiner selects NO in S32, registration of the new specialized protocol is deferred.

Even when the new specialized protocol is registered, the original protocol (standard protocol, specialized protocol) is maintained. Thus, a plurality of specialized protocols of different versions may be generated. During a version-update, the measurement value and the examination value are transferred.

FIG. 9 shows a specific example of a part of S24 described above. The illustrated content assumes the specialized protocol. In S40, the protocol execution controller sets an execution condition in the ultrasound diagnostic apparatus. When it is judged that the reference image is to be displayed in S42, the reference image is displayed in S44. When it is judged that the measurement value graph is to be displayed in S46, the measurement value graph is displayed in S48. When it is judged that the examination value graph is to be displayed in S50, the examination value graph is displayed in S52. Presence/absence of the display of each display element may be designated in advance with a flag or the like.

According to the embodiment described above, the standard protocol information is corrected by the protocol corrector, and the specialized protocol information is automatically generated. Then, during the execution of the protocol, the standard protocol or the specialized protocol may be selected. That is, the standard protocol may be applied to a new subject of examination, and, for a subject of examination for which the specialized protocol is generated, the specialized protocol may be applied, to further assist the ultrasound examination.

1. An ultrasound diagnostic apparatus comprising:
a processor, wherein the processor is configured to:
correct standard protocol information for executing a standard protocol which is a standard examination protocol, to generate specialized protocol information for executing a specialized protocol which is an examination protocol for a particular subject of examination;
sequentially set a plurality of execution conditions based on the standard protocol information during a course of sequential execution of a sequence of processes of the standard protocol, when the standard protocol is selected; and
sequentially set a plurality of execution conditions based on the specialized protocol information during a course of sequential execution of a sequence of processes of the specialized protocol, when the specialized protocol is selected.

2. The ultrasound diagnostic apparatus according to claim 1, wherein
the processor is further configured to:
associate, with respect to the sequence of processes of the specialized protocol and as a plurality of reference images, a plurality of ultrasound images acquired by the execution of the sequence of processes; and
execute, during the course of sequential execution of the sequence of processes of the specialized protocol, display control to sequentially display the plurality of reference images associated with the sequence of processes.

3. The ultrasound diagnostic apparatus according to claim 1, wherein
the processor is further configured to:
associate, with respect to a process with measurement in the specialized protocol, a measurement value acquired by execution of the process with measurement; and
execute, during execution of the process with measurement in the specialized protocol, display control to display one or a plurality of measurement values of the past associated with the process with measurement.

4. The ultrasound diagnostic apparatus according to claim 3, wherein
the processor is further configured to execute the display control to display the plurality of measurement values of the past as a measurement value graph.

5. The ultrasound diagnostic apparatus according to claim 1, wherein
the processor is further configured to:
associate, with respect to the specialized protocol, a plurality of examination values acquired in a time sequential order, from the particular subject of examination; and
execute, during execution of the specialized protocol, display control to display an examination value graph showing the plurality of examination values associated with the specialized protocol.

6. The ultrasound diagnostic apparatus according to claim 1, wherein
the standard protocol information includes a plurality of sets of process information for executing the plurality of processes of the standard protocol, and
the processor has:
a function to correct content of a set of process information which is a correction target among the plurality of sets of process information; and
a function to add a new set of process information to the plurality of sets of process information.

7. A method of assisting ultrasound examination, the method comprising:
correcting standard protocol information for executing a standard protocol which is a standard examination protocol, to generate specialized protocol information for executing a specialized protocol which is an examination protocol for a particular subject of examination;
sequentially setting a plurality of execution conditions based on the standard protocol information during a course of sequential execution of a sequence of processes of the standard protocol, when the standard protocol is selected; and
sequentially setting a plurality of execution conditions based on the specialized protocol information and displaying reference information acquired from the particular subject of examination and associated with the specialized protocol, during a course of sequential execution of a sequence of processes of the specialized protocol, when the specialized protocol is selected.
